(19) United States
(12) Patent Application Publication
(10) Pub. No.: US 2022/0071548 A1
(43) Pub. Date: Mar. 10, 2022

(54) BIOFEEDBACK BASED SYSTEM AND METHOD FOR PATIENT ASSESSMENT AND TREATMENT

(71) Applicant: Parameshwaran Ramakrishnan, Bothell, WA (US)

(72) Inventor: Parameshwaran Ramakrishnan, Bothell, WA (US)

(21) Appl. No.: 17/469,437

(22) Filed: Sep. 8, 2021

Related U.S. Application Data
(60) Provisional application No. 63/076,449, filed on Sep. 10, 2020.

(51) Int. Cl. A61B 5/374; A61B 5/31; A61B 5/375; A61B 5/313; A61B 5/397; A61B 5/00; A61B 5/16

(52) U.S. Cl. CPC A61B 5/374; A61B 5/31; A61B 5/375; A61B 5/313; A61B 2560/04; A61B 5/6803; A61B 5/165; A61B 5/4836; A61B 5/397

(57) ABSTRACT

A technique for using biofeedback to facilitate and quantify accurate patient assessments includes receiving brainwave activity readings of a provider and a recipient(s) before, during, and post an interaction between the provider and the recipient(s) for a procedure. The brainwave readings being indicative of the state of mind of the provider and the recipient(s). The provider performs a therapeutic or knowledge transfer to the patient(s)/recipient(s) and evaluates the impact/efficacy of the procedure on them. The system correlates the collected brainwave activity signals of the provider and the recipient(s) to determine the efficacy of the therapeutic or knowledge transfer procedure. The brainwave activity signals are collected using a headgear based on an electroencephalogram system and an electromyography system. The electromyography system positions electromyography sensors to measure scalp muscle movements of the provider and recipient(s) and remove the effects of scalp muscle movements from the electroencephalogram readings to provide true brainwave readings.

### CROSS-REFERENCE TO RELATED APPLICATION

This application claims priority to U.S. Provisional Application Ser. No. 63/076,449 filed on Sep. 10, 2020 and entitled “BIOFEEDBACK AND PATIENT ASSESSMENT AND TREATMENT SYSTEM AND METHOD”, the complete disclosure of which, in its entirety, is herein incorporated by reference.

### FIELD OF INVENTION

This invention relates to biofeedback systems, including a framework, system, and method for using biofeedback to facilitate and quantify accurate patient assessment and treatment.

### BACKGROUND

Empathic listening is a skill used by clinical chaplains/spiritual care providers to connect with patients on an emotional level during therapy sessions or therapeutic procedures, other types of interpersonal interactions, and mental healthcare services. During such sessions, it is not uncommon for patients to experience a “magical” and even “spiritual” healing experience during which their feelings of pain and loss may be fully understood and released.

The healing outcomes during such empathic listening sessions are evidenced when the patient becomes emotionally vulnerable, speaking his/her truth. Other objective pieces of evidence of the patient's spiritual healing such as goosebumps and increased skin conduction are measurable. However, they are indirect measurements of the provider's efficacy in empathic listening/spiritual care and assessment of the patient's mental distress.

Currently, there is no data-driven understanding of the processes and mechanisms that allow such empathic listening healing to manifest. For example, in schools of western medicine, there is a lack of technical coursework available to teach and explain the practice of this topic. Also, in the spiritual and/or religious realms, healing is often deemed as provided by the Divine.

Accordingly, there is a need for a system and method that provides a data-driven understanding of the processes and mechanisms that contribute to empathic listening and healing. There is also a need for a system that provides data-driven training on the topic of empathic listening and healing for healthcare practitioners.

Further, at workplaces and industries, the state of the mind of the individuals or workers working there is required to be monitored, else, it may lead to accidents in case they are in an undesired state of mind. There is, therefore, a need for a system and method that may allow a skilled person to care, while monitoring and assessing the state of mind of individuals who may be in an undesired state of mind due to workload or other reasons, so that the individuals may get some rest to refresh, and the concerned skilled person may be notified to take care of the situation.

### SUMMARY

An embodiment of the present disclosure relates to a system for using biofeedback to facilitate and quantify accurate patient assessments. The system may include a processor operatively coupled with a memory, storing a set of instructions, which may be processed by the processor. The processor may receive brainwave activity signals, before, during, and post a therapeutic/knowledge transfer procedure, from a first set of sensors configured in a primary headgear. The first set of brainwave activity signals may be associated with a provider (also known as knowledge provider or clinician, herein). The brainwave activity signals, in this first set, may be indicative of the empathic listening E/desired state of mind of the provider. The processor may further receive brainwave activity signals, before, during, and post the therapeutic/knowledge transfer procedure, from a second set of sensors configured in one or more secondary headgear(s). The second set of brainwave activity signals may be associated with one or more recipient(s) (also referred to as patient or knowledge seeker, herein). During the procedure, the provider may perform a therapeutic/knowledge transfer procedure to evaluate the impact on the recipient(s). The processor may correlate the first and second set of brainwave activity signals to determine the efficacy of the therapeutic/knowledge transfer procedure performed by the provider on the recipient(s).

Another embodiment of the present disclosure relates to a method. The method may include a step of receiving brainwave activity signals, before, during, and post a therapeutic/knowledge transfer procedure, from a first set of sensors configured in a primary headgear. The first set of brainwave activity signals may be associated with a provider, and the brainwave activity signals may be indicative of the empathic listening E/desired state of mind of the provider. The method may further include a step of receiving brainwave activity signals, before, during, and post the procedure, from a second set of sensors configured in one or more secondary headgear(s). The second set of brainwave activity signals may be associated with one or more recipient(s). During the procedure, the provider may perform a therapeutic/knowledge transfer impact on the recipient(s). The method may further include a step of correlating the first and second set of brainwave activity signals to determine the efficacy of the therapeutic/knowledge transfer procedure performed by the provider on the recipient(s).

### BRIEF DESCRIPTION OF THE DRAWINGS

Other objects, features, and characteristics of the present invention as well as the methods of operation and functions of the related elements of structure, and the combination of parts and economies of manufacture, will become more apparent upon consideration of the following description and the appended claims with reference to the accompanying drawings, all of which form a part of this specification. None of the drawings are to scale unless specifically stated otherwise.

FIG. 1 shows an overview of biofeedback and patient assessment system in accordance with exemplary embodiments hereof.

FIGS. 2A-2C show example brainwave activity data in accordance with exemplary embodiments hereof.

FIG. 3 shows example steps taken by biofeedback and patient assessment system in accordance with exemplary embodiments hereof.

FIG. 4 shows aspects of biofeedback and patient assessment system in accordance with exemplary embodiments hereof.

FIGS. 5A and 5B show example complete EEG graphs of a clinician (provider P) and a patient (recipient R), respectively, before, during, and post an empathic listening/healing procedure in accordance with exemplary embodiments hereof.

FIGS. 5C and 5D show normal and enlarged EEG graphs of the clinician, before the empathic listening/healing procedure in accordance with exemplary embodiments hereof.

FIGS. 5E and 5F show normal and enlarged EEG graphs of the patient, before the empathic

listening/healing procedure in accordance with exemplary embodiments hereof.

FIGS. 5G and 5H show normal and enlarged EEG graphs of the clinician, during the empathic listening/healing procedure in accordance with exemplary embodiments hereof.

FIGS. 51 and 5J show normal and enlarged EEG graphs of the patient, during the empathic listening/healing procedure in accordance with exemplary embodiments hereof.

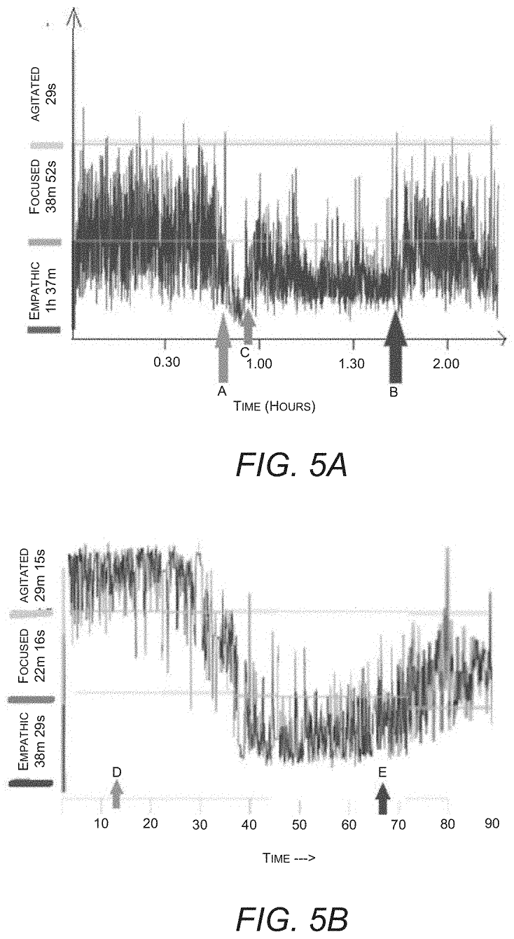

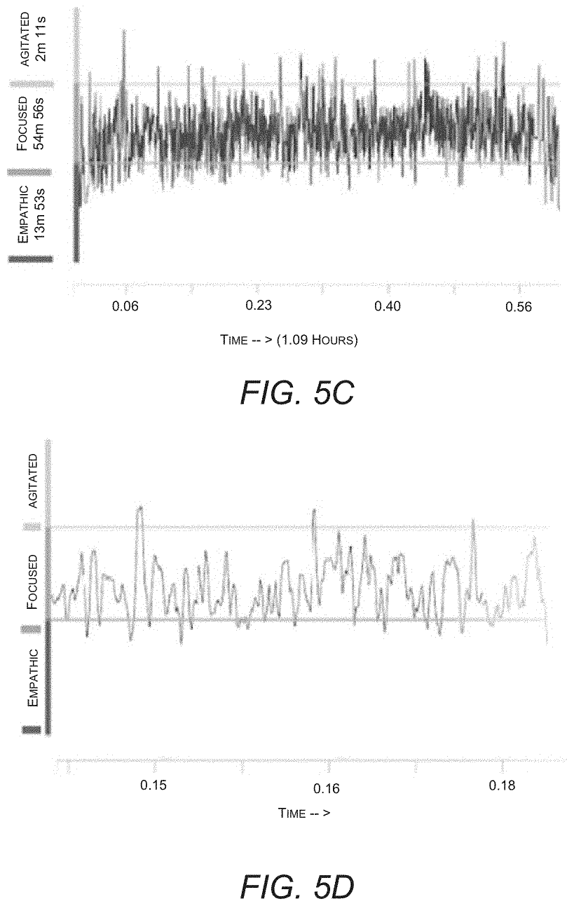

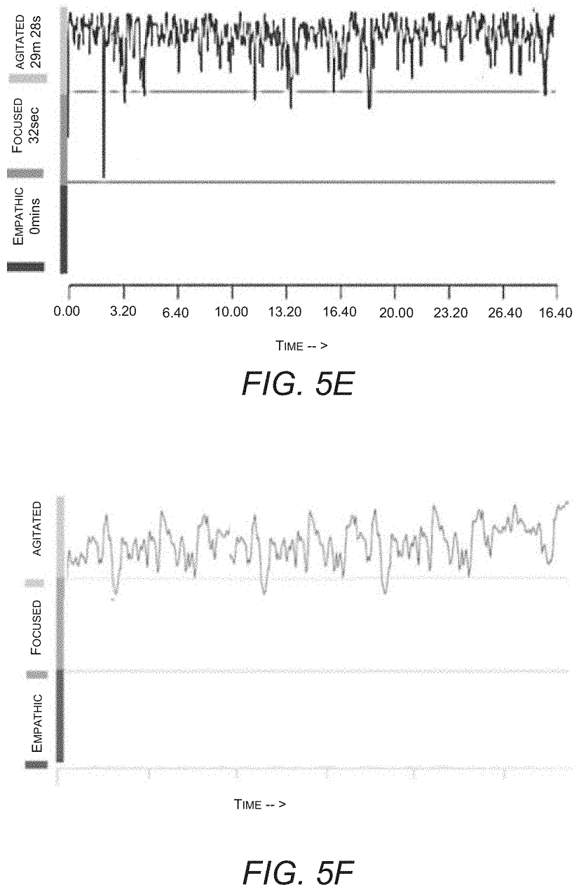

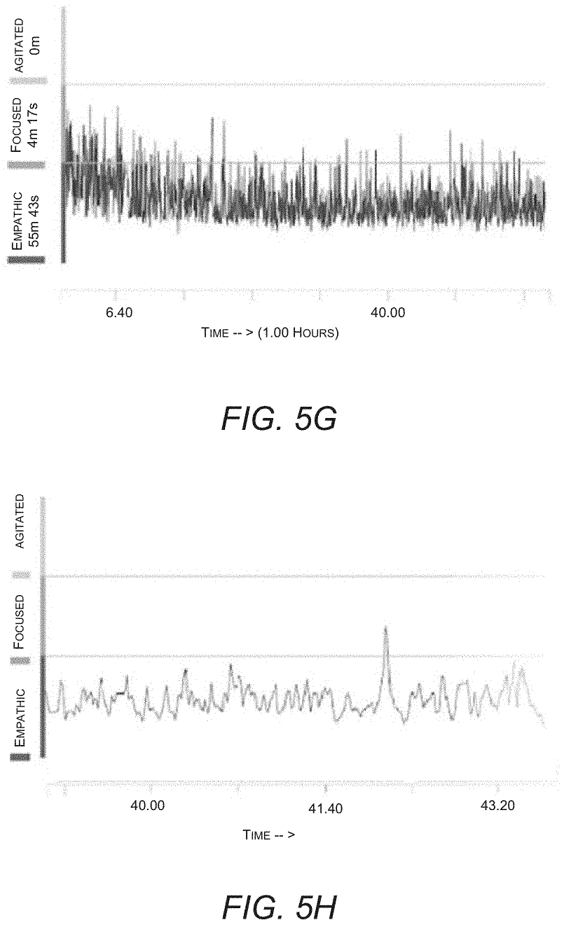

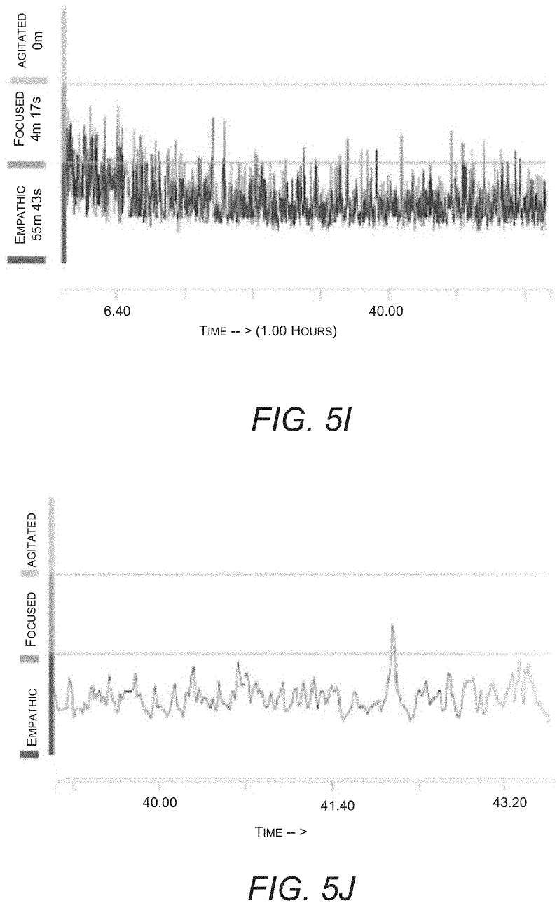

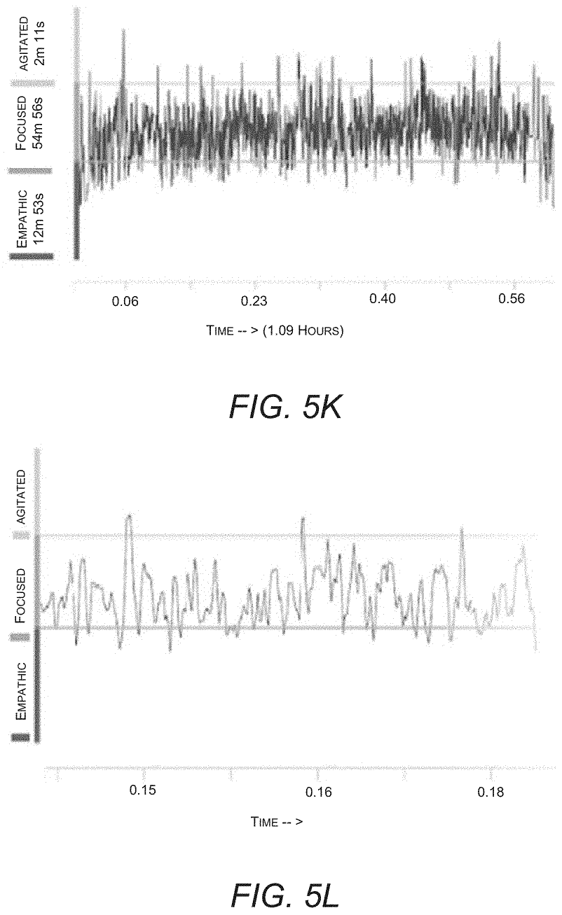

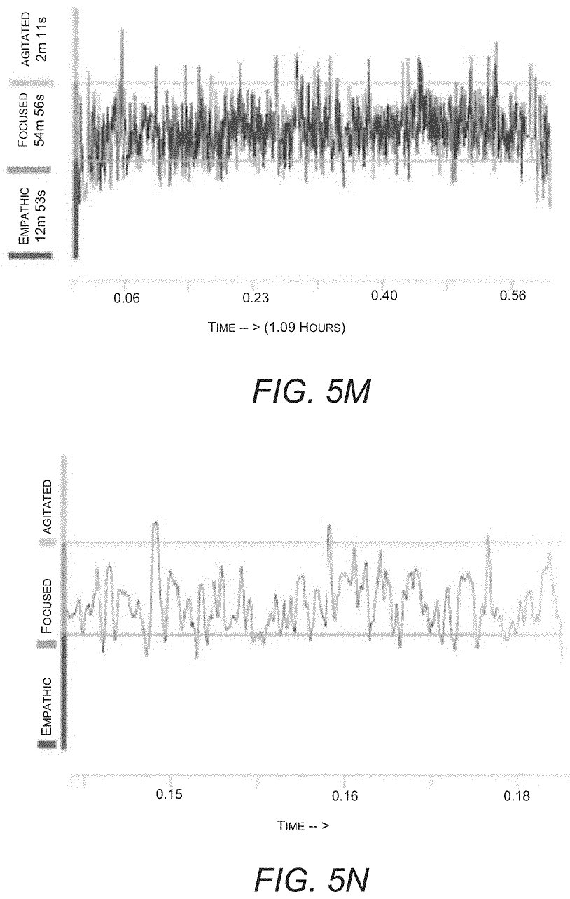

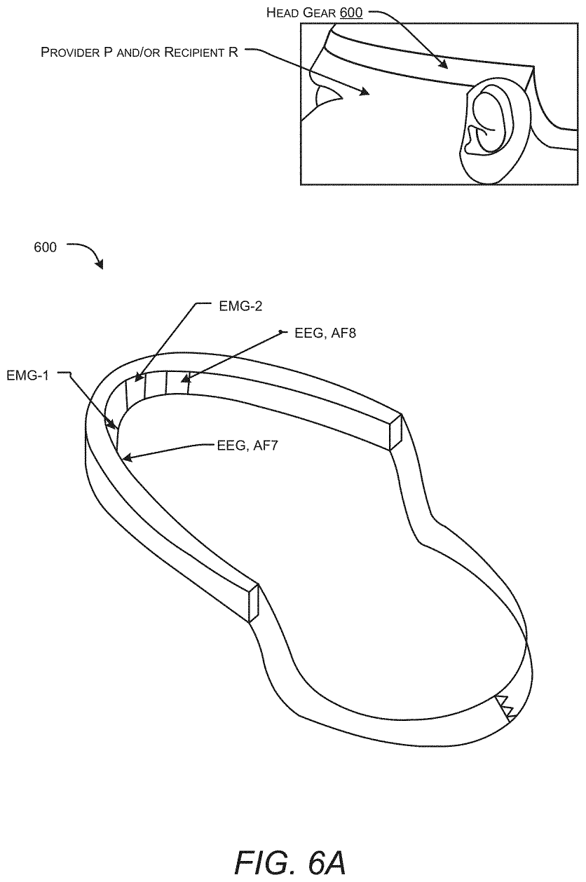

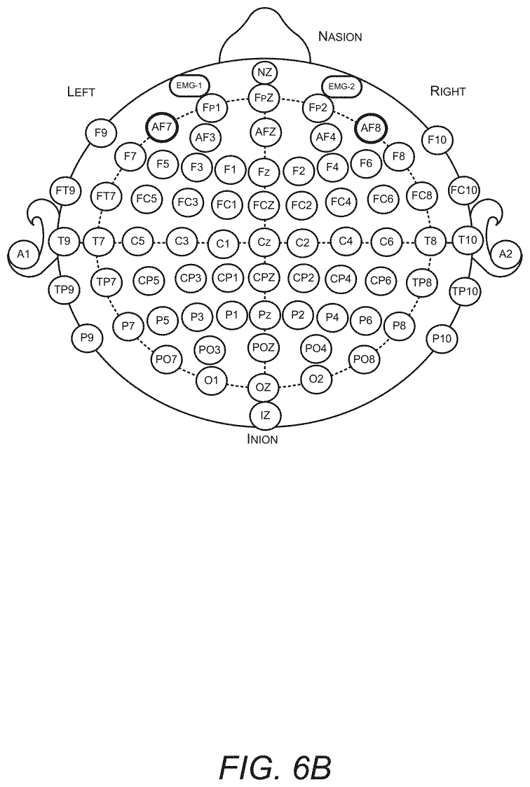

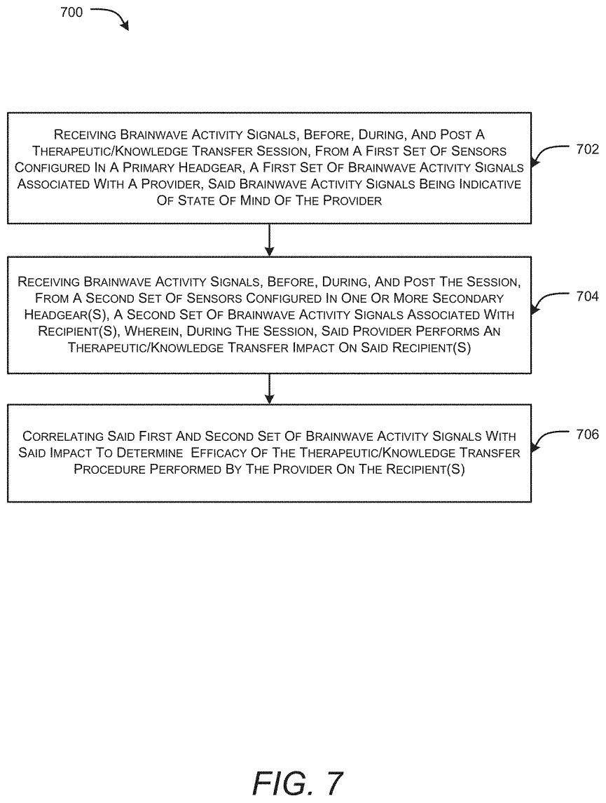

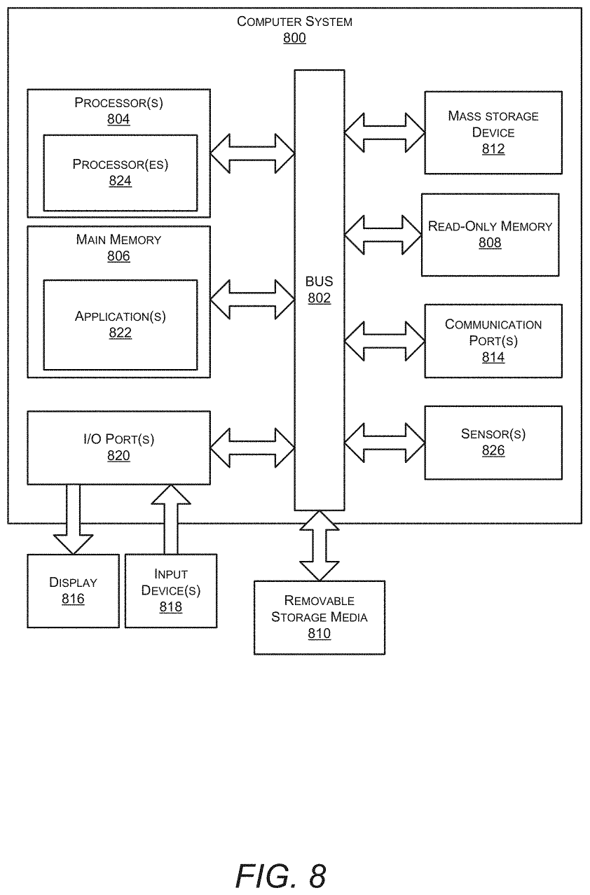

FIGS. 5K and 5L show normal and enlarged EEG graphs of the clinician, after the empathic listening/healing procedure in accordance with exemplary embodiments hereof.

FIGS. 5M and 5N show normal and enlarged EEG graphs of the patient, after the empathic listening/healing procedure in accordance with exemplary embodiments hereof.

FIG. 6A shows an example EEG system as headgear for capturing brain activity data of provider P and recipient R in accordance with exemplary embodiments hereof.

FIG. 6B shows an example anatomical location on the head of the provider P or recipient R where sensors of the EEG/EMG system or headgear of FIG. 6A is required to be positioned.

FIG. 7 shows an example flow diagram of biofeedback and patient assessment method in accordance with exemplary embodiments hereof.

FIG. 8 depicts aspects of computing and computer devices in accordance with exemplary embodiments hereof.

### DETAILED DESCRIPTION

As used herein, unless stated otherwise, the following terms and abbreviations have the following meanings.

The term provider P in the present disclosure will be collectively referred to any person who provides care and/or teaching to a recipient(s) R. For example, a provider may include (without limitation) a clinician, healthcare provider, knowledge provider, dietician, therapist, psychiatrist, doctor, nurse, medical assistant, chaplain, priest, rabi, teacher, counselor, any other type of care provider and any combination thereof. In addition, while this specification may use the term provider P predominantly as referring to a person or clinician who may provide mental healthcare, it is understood that a provider P may provide any type of care to a recipient(s) R. Further, the term recipient(s) R predominantly as referring to a patient who may receive the mental healthcare it is understood that a recipient(s) R may receive any type of care from a provider P.

The term recipient(s) R in the present disclosure will be collectively referred to anyone who may receive care and/or teaching and/or knowledge from a provider P. A recipient(s) R may include an individual or a group comprising patient, knowledge seeker, and the likes. The care may include (without limitation) counseling, therapy, assessment, diagnosis, treatment, other types of care, and any combination thereof.

Embodiments of the present disclosure relate generally to a framework, system, and method for using biofeedback to facilitate and quantify accurate patient assessments. According to exemplary embodiments herein, the present disclosure provides a system and method that collects brainwave activity readings of a provider P before, during, and post an interaction between the provider P and a recipient(s) R. The interaction may be related to a therapeutic procedure or a knowledge transfer procedure, but not limited to the likes. The therapeutic procedure may be any or a combination of counseling, assessment, diagnosis, therapy, and treatment undertaken by the provider P on the recipient(s) R. The knowledge transfer procedure may be a transfer of any knowledge shared by the provider P (knowledge provider) to the recipient(s) R (knowledge seeker).

In some embodiments, the system also may collect brainwave activity readings of the recipient(s) R before, during, and post the same interaction. The brainwave activity readings may indicate whether the provider P and/or the recipient(s) R are in an empathic E mental state (most preferred/most desired), a focused-attentive F mental state (preferred/desired) or an agitated A (unpreferred/undesired) mental state. The system also may collect information regarding the providers' P assessment and/or diagnosis of the recipient(s) R during the interaction. The system may also collect information relating to the efficacy (or accuracy) of the providers' P assessment and/or diagnosis (e.g., as determined after follow-up tests), and may correlate the accuracy information with the collected brainwave activity information to identify data patterns. The data patterns may be indicative of the states of mind through which the recipient(s) R and/or the provider P have transitioned through their interaction. For example, it may be found that a provider's assessment and/or diagnosis of a recipient(s) R is more efficient when the provider P and the recipient(s) R are in an empathic E mental state during their interaction. In some embodiments, the system may assign a grade or score (e.g., using a Likert scale) to an assessment and/or a diagnosis such as (i) strongly efficient, (ii) fairly efficient, (iii) fairly inefficient, (iv) strongly inefficient, and/or (v) uncertain. Other types of qualifying methods also may be used.

The system may also correlate the success (or lack of success) of ensuing recipient(s) R treatment plans and/or knowledge transfer with the mental states of the provider P and/or the recipient(s) R (taken before, during, and post the assessment and/or diagnosis and/or knowledge transfer of the recipient(s) R. The correlation may indicate emotional/empathic connection and/or synchronization of the recipient(s) R with the provider P.

Post the procedure, the system may obtain information associated with a treatment/follow-up plan provided by the provider P to the recipient(s) R and may allow evaluation of the recipient(s) R at subsequent desired time intervals (follow-up procedure) to determine the impact/efficacy of the treatment/follow-up plan based on analysis of the brainwave activity signals associated with the recipient(s) R at such respective time intervals.

The system may also gather head muscle movement signals using Electromyography (EMG) sensors (or similar), which may produce false-positive brainwaves in the EEG sensors. The gathered head muscle movement signals may be used to mathematically remove the effects of scalp muscle movements from the EEG readings, thereby eliminating any false-positive brainwaves to produce true brain waves for correlation. The above and additional functionalities of the system will be described in detail hereinafter.

Those skilled in the art would appreciate that the system and method of the present disclosure may also be extended to have wider applications in the field of education, manufacturing and service industries, transport, railways, aviation, navigations, governmental agencies, and the likes, and all such embodiments are well within the scope of the present disclosure.

In the industrial sector, provider P may be the chief officer, and the recipient(s) R may be the subordinates. The system may allow the chief to monitor and assess the state of mind of the subordinates due to work stress and may alert the subordinates to refresh or to get some rest. It can also notify the chief so that they can take care of the situation. The chief can also regularly monitor the state of the mind of the subordinates and take necessary steps to prevent accidents if they find any deviation in their state of the mind.

In the education sector, the provider P may be a teacher and the recipient(s) R may be the students.

In the transport sector, the provider P may be chief supervisors responsible for a fleet of vehicles such as bus/car/truck/other heavy vehicles, located at a control room, and the recipient(s) R may be drivers.

In the railway sector, the provider P may be chief supervisors at a control room, and the recipient(s) R may be the loco pilots.

In the aviation sector, the provider P may be a chief supervisor in a control room, and the recipient(s) R may be a captain/pilot.

In the navigation sector, the provider P may be captain and the recipient(s) R may be helmsman and all the crew members.

In the government sector, the provider P may be a chief, and the recipient(s) R may be the subordinates.

In the defense sector, the provider P may be captains, and the recipient(s) R may be soldiers.

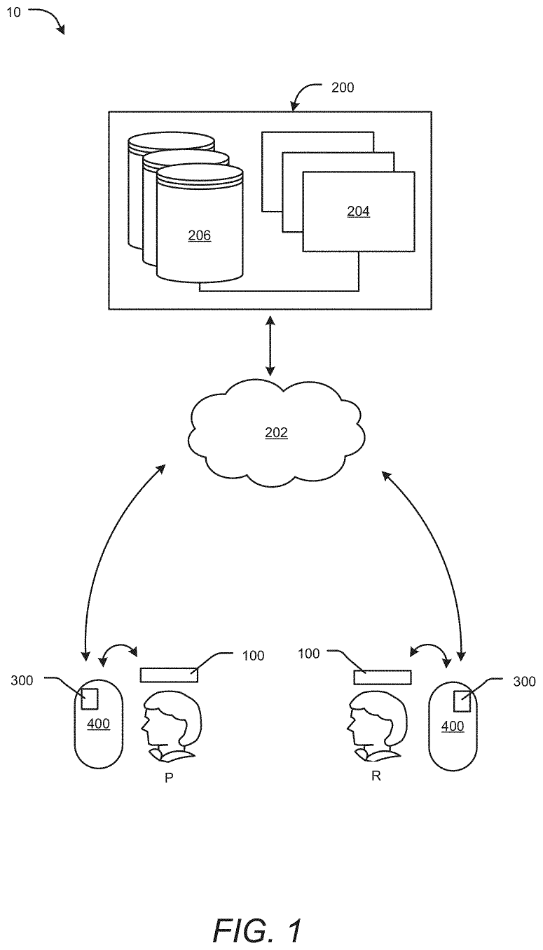

FIG. 1 shows an overview of an exemplary framework for a biofeedback and patient assessment system 10 (also referred to herein as simply the system 10) according to exemplary embodiments herein. As shown, the biofeedback and patient assessment system 10 may include one or more biofeedback systems 100 that may interface with a backend system 200. System 10 also may include an application 300 (e.g., software residing on a local computer, a mobile application or “app”, a website viewed using a browser, or other types of applications) that may be used to interface with the biofeedback system 100 and/or the backend system 200. The interface may include a network 202 (e.g., the Internet, LAN, WAN, etc.), wireless communication systems, cellular communication systems, telephony, or other types of communication systems or protocols. System 10 may also include other systems, elements, and components as required by system 10 to fulfill its functionalities.

System 10 may be accessed by multiple users including provider P and/or recipient(s) R via network 202 and using the application 300 running on one or more computing devices 400 associated with the provider P and recipient(s) R. The mobile devices may include but are not limited to smartphones, tablet computers, laptops, desktop computers, and mobile media players.

In some embodiments, the backend system 200 may include a cloud platform (e.g., one or more backend servers), one or more local controllers, or any combination thereof. In some embodiments, the backend system 200 includes a cloud platform that interfaces with one or more local controllers. For example, administrators of system 10 may interface with system 10 via a local controller in communication with a cloud platform. The backend system 200 may include a plurality of applications 204 and databases 206 as will be described in other sections.

Application 300 includes a graphical user interface (GUI) that may be presented on the device's 400 display and that includes controls (e.g., touchscreen and/or mechanical buttons, etc.) that a user may activate to interact with the system 10. For example, the GUI may include controls and/or other mechanisms that enable the user to interface with the system 10 to log into the system 10, pair the biofeedback system 100 to device 400, receive information from the biofeedback system 100, present the information on the display of the device 400, upload the information to the backend system 200, receive information from the backend system 200, etc. In some embodiments, application 300 may present instructions, wizards, and/or other types of guidance to the users via the GUI. In addition, application 300 may accommodate any language.

In some embodiments, some of the functionalities of system 10 may be performed by the backend system 200, and some of the functionalities of system 10 may be performed by application 300. It also is understood that some of the functionalities of system 10 may be performed by the backend system 200 and the application 300 in combination, and/or by both the backend system 200 and application 300 depending on the circumstances.

It will be appreciated by a person of ordinary skill in the art that the system 10 may be used in conjunction with any type of interaction between one provider P and one or more recipient(s) R, and that the interactions may take place in any setting and under any circumstances and/or conditions. For example, system 10 may be used during therapy procedures with or without the use of therapeutic medication. For example, the portable EEG-EMG-feedback and spiritually-augmented insight-oriented psychotherapy provided by system 10 may enhance the efficacy of ketamine (Entheogen)-assisted psychotherapy as well as any other types of psychotherapies.

In some embodiments, the biofeedback system 100 includes an electroencephalogram (EEG)-electromyogram (EMG) system. As is known in the art, EEG is an electrophysiological monitoring method that measures and records the electrical activity of the brain. Clinically, EEG refers to the recording of the brain's spontaneous electrical activity and EMG refers to the electrical activity caused by scalp muscles' movements over a period of time. The EEG-EMG system includes electrodes that are attached to the provider's P/recipient(s)′ R scalp that sense the voltage fluctuations (also referred to as “brainwaves”) resulting from ionic current within the neurons of the brain.

FIG. 6A shows an example EEG-EMG system as a headgear 600 for capturing brain activity and scalp muscles' activity data of provider P and recipient(s) R in accordance with exemplary embodiments hereof.

FIG. 6B shows example anatomical locations on the head of the provider P or recipient(s) R where sensors of the EEG-EMG system or headgear 600 of FIG. 6A are required to be positioned thereon to capture brainwaves and scalp muscles' movement signals.

In some embodiments, the EEG-EMG system provides scalp electrodes at locations specified by the International 10-20 system. In other embodiments, the EEG system provides scalp electrodes at one or more subsets of the locations specified by the International 10-20 system (e.g., by using an EEG headgear 600 shown in FIG. 6B. It is understood that the EEG system may provide any type and number of scalp electrode(s) at any location for the brainwave measurements and that the scope of system 10 is not limited in any way by the number and/or location of the electrodes used. The headgear 600 with anatomical locations of the EEG sensors corresponding to AF7 and AF8, and EMG sensors corresponding to Fp1 and Fp2 leads of the 0-20 system as shown in FIG. 6A. In the inset of FIG. 6A, demonstration of how the headgear 600 is worn by provider P or recipient(s) R, across the forehead.

In some embodiments, the EEG system is controlled using the application 300 running on the electronic device 400 (e.g., a smartphone or tablet computer). Application 300 may present a graphical user interface (GUI) that enables the user to configure the EEG system, to take brainwave measurements over a period of time, and to plot the consolidated (A, F, E) vs. components (alpha “a”, beta “b”, gamma “g”, theta “t”, delta “d”) of brain waves.

The brainwaves measured by EEG systems are typically characterized by their amplitudes and frequencies and are subsequently classified into five main types as described below.

1. Gamma (g) brainwaves: These brainwaves have a frequency of 32 Hz-100 Hz and are associated empathic listening and transcendental or beyond-mindfulness meditative state of mind.

2. Beta (b) brainwaves: These brainwaves have a frequency of 13 Hz-32 Hz and are associated with alert, normal alert consciousness, active thinking, and also pathological states of mind such as anxiety, fearfulness, etc.

3. Alpha (a) brainwaves: These brainwaves have a frequency of 8 Hz-13 Hz and are associated with physically and mentally relaxed states or the state of Focused-Attention in meditation.

4. Theta (t) brainwaves: These brainwaves have a frequency of 4 Hz-8 Hz and are associated with reduced conscious-awareness, sleep, dreams, and also creativity and insightful or mindful-meditation.

5. Delta (d) brainwaves: These brainwaves have a frequency of 0.5 Hz-4 Hz and are associated with deep and dream-less sleep and also in mindful and beyond mindfulness meditation.

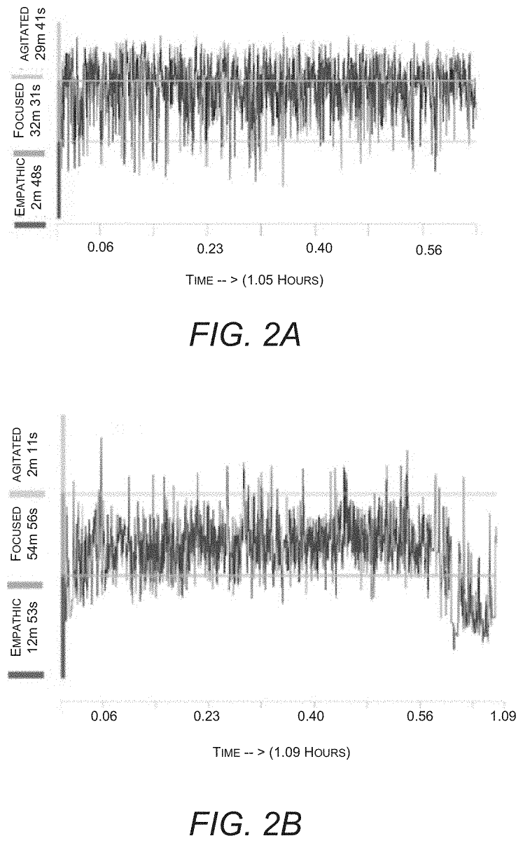

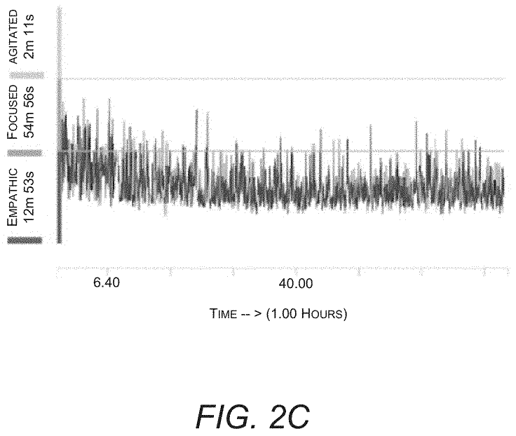

During an EEG measurement, the various brainwaves may be measured, combined into a single EEG trace, and plotted vs. time. Examples of this are shown in FIGS. 2A, 2B, and 2C. FIG. 2A shows a consolidated EEG trace comprising mostly with Beta brainwaves component. For the purposes of this specification, this trace will be classified as mainly “agitated A”. FIG. 2B shows a consolidated EEG trace comprising mostly Alpha brainwaves component. For the purposes of this specification, this trace will be classified as mainly “focused-attention F” as shown and associated with a mental state that may be generally relaxed and mindful-meditative state. FIG. 2C shows a consolidated EEG trace comprising mostly Theta, Delta, and Gamma brainwaves component. For the purposes of this specification, this trace will be classified as mainly “empathic listening E” as shown and associated with a mental state that may be generally termed as beyond-mindfulness or transcendental meditative state.

The EEG systems or headgear of the biofeedback system 100 include Electromyography (EMG) sensors to measure scalp muscle movements of the user and mathematically remove the effects of scalp muscle movements from the EEG readings to provide true brainwave readings.

In general, system 10 may be used by a provider P and/or recipient(s) R before, during, and post a provider-recipient interaction. For example, in some embodiments, the following steps be taken (without limitation) during the use of the system 10.

1. Prior to providing a recipient(s) R with mental healthcare, a provider P may be configured with the EEG system adapted to measure and record the provider's brainwaves for analysis. For example, a provider P may wear an EEG headgear 600 coupled to the EEG system and control via the application 300 running on the provider's device 400 (e.g., smartphone or tablet computer).

2. The provider P first take a baseline EEG measurement of his/her brainwaves, and then depending on the readings, may then relax (e.g., meditate) until the EEG readings drop from an agitated A or focused-attention F state to an empathic/meditative E state. Application 300 may notify the provider P when an empathic E state has been reached and held for a predetermined amount of time (e.g., 90% empathic E for 2-5 minutes).

3. Once an empathic listening E state of mind has been reached and held, the provider P may begin to interact with the recipient(s) R. The provider P may continue to monitor his/her brainwave activity during the interaction via application 300 and attempts to maintain his/her E mental state throughout. This allows the provider P to provide empathic listening, deep understanding, etc. to the recipient(s) R. The application 300 may notify the provider P if his/her brainwave activity emerges out of the desired empathic listening E threshold so that he/she may take necessary steps (e.g., to meditate again) to enter back into the empathic meditative E state.

4. During the provider's P interaction with the recipient(s) R, the provider's P goal is to transfer his/her empathic/meditative E mental state to the recipient(s) R, allowing the recipient R to become calm, safe, emotionally vulnerable, and able to speak his/her truth. This empathic/meditative E state of both the provider P and the recipient(s) R facilitates an emotional connection between the two, resulting in a potentially more accurate assessment and/or diagnosis of the recipient R by the provider P, and subsequently, a potentially more successful treatment plan.

5. Post the provider's P interaction with the recipient R, the information associated with a treatment/follow-up plan provided by the provider P to the recipient(s) R may be obtained, and evaluation of the recipient at subsequent desired time intervals (follow-up procedures) may be initiated to determine the impact/efficacy of the treatment/follow-up plan based on analysis of the brainwave activity signals associated with the recipient(s) R at such respective time intervals.

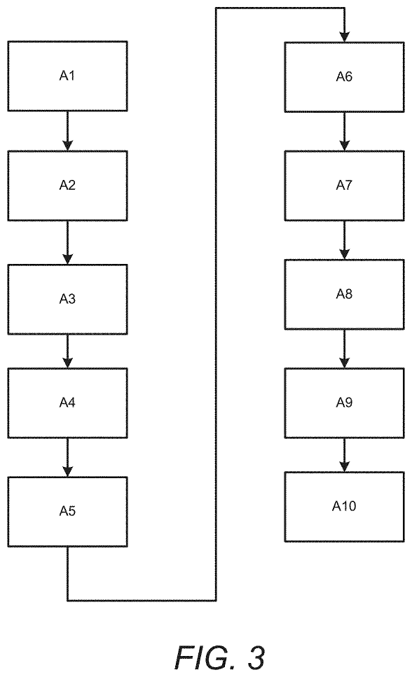

In some embodiments, system 10 may be used to gather and correlate data, and to quantify the data correlations to provide training opportunities for providers P. For example, in some embodiments as shown in FIG. 3, system 10 may perform the following steps (without limitation).

At step A1, the information regarding the provider's P brainwave activity and/or the recipient(s)′ R brainwave activity prior to, during, and post interactions between the provider P and the recipient(s) R may be collected. For example, system 10 may measure and record the provider's P brainwave activity during the period of time immediately prior to an appointment with the recipient(s) R (to establish the provider's P baseline brainwave activity), during the appointment to record his/her brainwave activity while assessing the recipient(s) R, and directly after the appointment. This may include collecting information taken by the EEG system. In another example, system 10 also may measure and record the recipient(s)' R brainwave activity during the appointment. This information is stored, and time/date stamped.

At step A2, the information regarding any assessment and/or diagnosis of the recipient(s)' R medical condition made by provider P during the same interactions may be collected. Expanding on the example from above, system 10 may record information regarding the provider's P assessment and/or diagnosis of the recipient(s) R made during the appointment. This information may be provided to system 10 by provider P

At step A3, the information regarding the accuracy (and/or inaccuracy) of the provider's P assessment and/or diagnosis of the recipient's R medical condition from step A2 above. This information may be provided to system 10 as the information may become available, e.g., during subsequent or follow-up provider P-recipient(s) R interactions. For example, it may be determined, given data received from follow-up tests, that the provider's initial assessment of the recipient R may have been accurate (or inaccurate).

At step A4, the accuracy (and/or inaccuracy) of the provider's assessment and/or diagnosis-collected in step A3 may be correlated with the brainwave activity information of the provider P and/or the recipient R collected at step A1.

At step A5, patterns in the correlations made in step A4 may be identified. For example, system 10 may find patterns of accurate diagnosis made during interactions when the provider's and/or the recipient's brainwave activities were measured to be in a predominantly empathic E state. In another example, system 10 may find patterns of inaccurate diagnosis made during interactions when either the provider P and/or the recipient R were shown to have brainwave activities that remained in the agitated A or focused-attentive F state.

At step A6, the information regarding the subsequent treatment plan(s) provided by the provider P and/or by other healthcare providers to the recipient(s) R (e.g., treatment plans based on the provider's diagnosis of the recipient R) may be collected.

At step A7, the information regarding the success and/or any problems associated with the treatment plans may be collected. This information may be provided to system 10 as the information may become available, e.g., during subsequent or follow-up provider P-recipient(s) R interactions, during interactions between the recipient R, or by other methods. For example, it may be determined, given information showing an improvement in the recipient's condition, that a treatment plan has been successful.

At step A8, the efficacy or accuracy of the treatment plans collected in step A7 with the brainwave activity information of the provider P and/or the recipient(s) R collected in step A1 may be correlated.

At step A9, patterns in the correlations made in step A8 may be identified. For example, system 10 may find patterns of successful treatment plans stemming from a diagnosis made when the provider's P and/or the recipient(s)′ R brainwave activities were measured to be in a predominantly empathic E state. In another example, system 10 may find patterns of unsuccessful treatment plans stemming from a diagnosis made when either the provider P and/or the recipient R were shown to have brainwave activities that were remained in the agitated A or focused-attentive F state.

Accordingly, at step A10, the information collected in steps A1 to A9 may be used and for educational purposes for providers P-in-training.

It is understood that the summary of steps described above is meant for demonstration and that the actual process may include additional steps not listed. It also is understood that not all of the listed steps may be taken, that any of the steps may be combined, and that any of the steps may be performed in any different order.

FIGS. 5A to 5N show example EEG graphs of the provider P as a clinician (doctor) and the recipient R as a patient, respectively, before, during, and post an empathic listening procedure provided by the clinician to the patient, in accordance with exemplary embodiments hereof.

The EEG graph for the clinician was recorded for 2.5 hours. FIG. 5A shows an example complete EEG graph of the clinician, before, during, and post the empathic listening procedure. FIGS. 5C and 5D show normal and enlarged EEG graphs of the clinician, before the empathic listening procedure. FIGS. 5G and 5H show normal and enlarged EEG graphs of the clinician, during the empathic listening procedure. FIGS. 5K and 5L show normal and enlarged EEG graphs of the clinician, after the empathic listening procedure.

The recorded EEG graph of the clinician includes the clinician's EEG recording during his/her clinical office works before point A. The EEG recording during his/her official work after visiting the patient is shown after point B. The duration between point A and point C in the EEG graph is the duration when the clinician spends few minutes in silent mediation to prepare himself to provide the empathic listening procedure (spiritual session) to the patient.

Referring to FIG. 5B, the EEG graph for the patient was recorded for approx. 90 minutes. FIGS. 5E and 5F show normal and enlarged EEG graphs of the patient, before the empathic listening procedure. FIGS. 5I and 5J show normal and enlarged EEG graphs of the patient, during the empathic listening procedure. FIGS. 5M and 5N show normal and enlarged EEG graphs of the patient, after the empathic listening procedure.

Referring to FIG. 5B, the recorded EEG graph of the patient includes the EEG recording during the empathic listening procedure between points D and E, which is approximately 45 minutes. The EEG recording before point D is before the patient's empathic listening procedure, which is about 10-12 minutes. The EEG recording after point E is after the patient's empathic listening procedure ended, which is about 30 minutes.

System 10 may correlate the EEG graph or brain wave activity signals of the clinician and patient as shown in FIGS. 5A to 5 N to determine the efficacy of the empathic listening procedure performed by the clinician on the patient. As can be inferred from the figures, the patient, as well as the clinician, were in focused-attention F/mindfulness or empathic or deeply meditative E/transcendental mental states during their interaction. Before the interaction, the patient was in an agitated A mental state, but during the interaction, the patient came into an empathic E mental state. Further, after the interaction, the patient remained in a desirable healed empathic E or focused-attention F mental state for some time, without returning to the agitated A mental state. Besides, the clinician remained either in the focused-attentive F and/or empathic E mental state before, during, and after the interaction. Further, the clinician centered himself with a brief prayerful or meditative session, just before the interaction. Thus, the system found the efficacy of the empathic listening procedure to be good as the state of mind of the patient during and after the course of the session as indicated by the respective brainwave activity signals matched with the brainwave activity signals of the clinician.

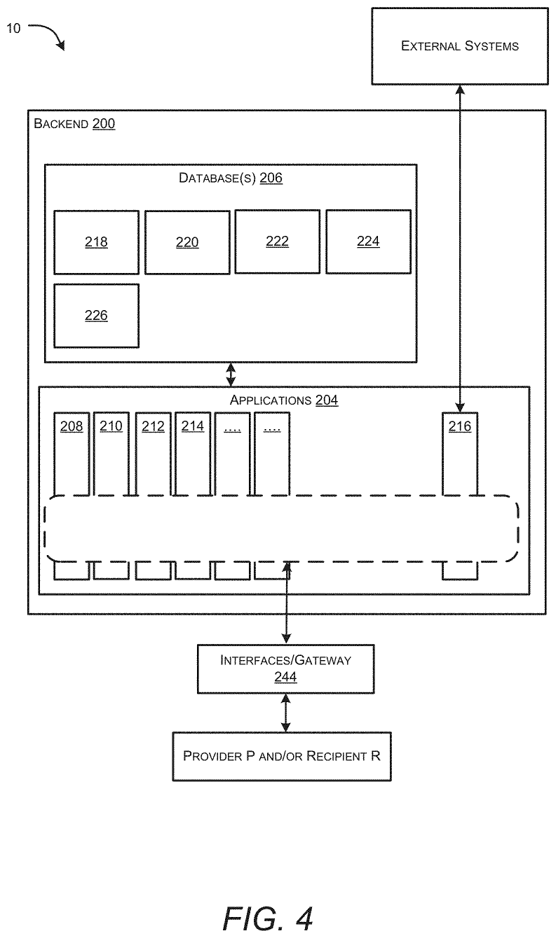

FIG. 4 shows aspects of exemplary biofeedback and patient assessment system 10 of FIG. 1. As shown, system 10 and backend system 200 comprises various internal applications 204 and one or more databases 206, described in greater detail below. The internal applications 204 may generally interact with one or more databases 206 and the data stored therein.

The database(s) 206 may comprise one or more separate or integrated databases, at least some of which may be distributed. The database(s) 206 may be implemented in any manner, and, when made up of more than one database, the various databases need not all be implemented in the same way. It should be appreciated that the system is not limited by the nature or location of the database(s) 206 or by the manner in which they are implemented.

Each of the internal applications 204 may provide one or more services via an appropriate interface. Although shown as separate applications 204 for the sake of this description, it is appreciated that some or all of the various applications 204 may be combined. The various applications 204 may be implemented in any manner and need not all be implemented in the same way (e.g., using the same software languages, interfaces or protocols).

In some embodiments, the applications 204 may include one or more of the following applications 204 comprising an information input/output application(s) 208, an assessment and/or diagnosis data correlation application(s) 210, treatment plan correlation application(s) 212, data analysis application(s) 214, and a data reporting application(s) 216, but not limited the likes.

The information input/output application(s) 208 may input data from application 300, the device 400 and/or from other sources, and output data to application 300, the device 400 and/or to other sources. The assessment and/or diagnosis data correlation application(s) 210 may correlate brain activity data with assessment and/or diagnosis accuracy data as described herein. The treatment plan correlation application(s) 212 may correlate brain activity data with treatment plan success data as described herein. The data analysis application(s) 214 may analyze and find patterns of data in the assessment and/or diagnosis data correlations, and the treatment plan correlations as described herein. The data reporting application(s) 216 may generate any type of report regarding the use and/or functionalities of the system 10 including correlated data, data patterns, any other types of data and/or information, and any combination thereof.

Applications 204 also may include other applications and/or auxiliary applications (not shown). Those of ordinary skill in the art will appreciate and understand, upon reading this description, that the above list of applications is meant for demonstration and that system 10 may include other applications that may be necessary for system 10 to generally perform its functionalities as described in this specification. In addition, as should be appreciated, embodiments or implementations of system 10 need not include all of the applications listed, and that some or all of the applications may be optional. It is also understood that the scope of system 10 is not limited in any way by the applications that it may include.

In some embodiments, the database(s) 206 may include one or more of the following databases 206 comprising a brainwave activity data database(s) 218, an assessment and/or diagnosis database(s) 220, a correlated data database(s) 222, a data analysis database(s) 224, and a data report(s) database(s) 226, but not limited to the likes.

The brainwave activity data database(s) 218 may store any type of brainwave activity-related data received from the biofeedback system 100. The assessment and/or diagnosis database(s) 220 may store any type of information regarding any provider's assessment and/or diagnosis of any recipient R. The correlated data database(s) 222 may store any data and/or other types of information related to any correlation of data between assessments, diagnosis, treatment plans and/or recorded brainwave activities. The data analysis database(s) 224 may store any information regarding patterns of data found in any of the data correlations. The data report(s) database(s) 226 may store any reports of any kind generated by the system

It is understood that the above list of databases 218 to 226 is meant for demonstration and that system 10 may include some or all of the databases, and also may include additional databases as required. It is also understood that the scope of system 10 is not limited in any way by the databases that it may include.

Various applications 204 and databases 202 in the biofeedback and patient assessment system 10 may be accessible via an interface(s) 242. These interfaces 242 may be provided in the form of APIs or the like and made accessible to external users (e.g., provider P, recipient R, etc.) via one or more gateways and interfaces 244 (e.g., via a web-based application 300 and/or a mobile application 300 running on the user's personal device 400 such as a mobile phone, tablet computer, desktop computer, laptop computer, etc.).

It is understood that any aspect and/or element of any embodiment described herein or otherwise may be combined in any way to form new embodiments all of which are easily understood by a person of ordinary skill in the art and all of which are within the scope of the system 10.

FIG. 7 illustrates a flow diagram of the disclosed method 700 for biofeedback and patient assessment. At 702, method 700 may include the step of receiving brainwave activity signals associated with a provider P or knowledge provider, before, during, and post a therapeutic and/or knowledge transfer procedure. The first brainwave activity signals of the provider P may be collected from a first set of sensors configured in a primary headgear. The first brainwave activity signals being indicative of the state of mind of the provider P. At 704, method 700 may include the step of receiving brainwave activity signals associated with a recipient(s) R or a knowledge seeker, before, during, and post the procedure. The second brainwave activity signals of the recipient(s) R may be collected from a second set of sensors configured in one or more secondary headgear(s). The second brainwave activity signals being indicative of the state of mind of the recipient(s) R. The first and second set of brainwave activity signals at the steps 702 and 704 may be gathered either intermittently at defined intervals or may be obtained continuously before, during and after the therapeutic/knowledge transfer procedure.

During the procedure, the provider P may perform a therapeutic or knowledge transfer impact on the recipient(s) R or knowledge seeker. At 706, method 700 may include the step of correlating the first and second set of brainwave activity signals received at 702 and 704, with the impact to determine the efficacy of the therapeutic or knowledge transfer procedure performed by the provider P on the recipient(s) R.

The state of mind may be any part or a combination of calm mental state, also termed as empathetic listening E mental state (mindfulness and beyond mindfulness/transcendental state), focused and attentive F mental state, and an agitated A mental state. The efficacy of the therapeutic/knowledge transfer procedure may be higher when the recipient(s)' R state of mind during and after the course of the procedure as indicated by the respective brainwave activity signals matches the brainwave activity signals of the provider P.

Step 706 of correlating the first and second set of brainwave activity signals with the impact may enable the identification of data patterns indicative of the states of mind through which the recipient(s) R and/or the provider P have transitioned through the procedure. Further, the efficacy of the therapeutic or knowledge transfer procedure performed by the provider P on the recipient(s) R may be objectified on any or a combination of a grade, range, scale, value, or a score.

In some embodiments, the efficacy of the therapeutic/knowledge procedure may be determined based on one or more efficacy markers comprising any or a combination of an agitated A state of mind, a focused attention F state of mind, a mindful and empathic E/transcendental state of mind, and other finer categories. In the agitated A state of mind, predominantly high beta waves along with very low gamma & alpha waves may be found. In the focused attention F state of mind, predominantly high and sustained alpha waves may be found. In the mindful and empathic E state of mind, predominantly high delta and theta waves may be found. In the beyond-mindful and empathic/transcendental E state of mind, predominantly high beta and sustained gamma waves may be found. Further, the other finer categories may be based on a combination of any of Gamma brainwaves, Beta brainwaves, Alpha brainwaves, Theta brainwaves, and Delta brainwaves associated with the brain activity signals.

Further, post the provider's interaction with the recipient(s) R, method 700 may also include the step of obtaining information associated with a treatment/follow-up plan provided by the provider P to the recipient(s) R, and evaluating the recipient R at subsequent desired time intervals (follow-up procedure) to determine the impact/efficacy of the treatment/follow-up plan based on analysis of the brainwave activity signals associated with the recipient at such respective time intervals.

The services, mechanisms, operations, and acts shown and described above are implemented, at least in part, by software running on one or more computers or computer systems or devices. It should be appreciated that each user device is, or comprises a computer system.

Programs that implement such methods (as well as other types of data) may be stored and transmitted using a variety of media (e.g., computer-readable media) in a number of manners. Hard-wired circuitry or custom hardware may be used in place of, or in combination with, some or all of the software instructions that can implement the processes of various embodiments. Thus, various combinations of hardware and software may be used instead of software only.

One of ordinary skill in the art will readily appreciate and understand, upon reading this description, that the various processes described herein may be implemented by, e.g., appropriately programmed general-purpose computers, special purpose computers, and computing devices. One or more such computers or computing devices may be referred to as a computer system.

FIG. 8 is a schematic diagram of a computer system 800 upon which embodiments of the present disclosure may be implemented and carried out.

According to the present example, the computer system 800 includes a bus 802 (i.e., interconnect), one or more processors 804, one or more communications ports 814, a main memory 806, removable storage media 810, read-only memory 808, and mass storage 812. Communication port(s) 814 may be connected to one or more networks by way of which the computer system 800 may receive and/or transmit data.

The processor 804 may be operatively coupled to the read-only memory 808, storing a set of instructions, which when processed by the processor 804, may cause the processor 804 to receive brainwave activity signals associated with a provider P or knowledge provider, before, during, and post a therapeutic and/or knowledge transfer procedure. The first brainwave activity signals of the provider P may be collected from a first set of sensors configured in a primary headgear. The first brainwave activity signals being indicative of the state of mind of the provider P. Further, the execution of instructions by the processor 804, may cause the processor 804 to receive brainwave activity signals associated with a recipient(s) R or a knowledge seeker, before, during, and post the procedure. The second brainwave activity signals of the recipient(s) R may be collected from a second set of sensors configured in one or more secondary headgear(s). The second brainwave activity signals being indicative of the state of mind of the recipient(s) R. The first and second set of brainwave activity signals may be gathered either intermittently at defined intervals or may be obtained continuously before, during, and after the therapeutic/knowledge transfer procedure. Furthermore, the execution of instructions by the processor 804, may cause the processor 804 to correlate the first and second set of brainwave activity signals with the impact, which may enable the identification of data patterns indicative of the states of mind through which the recipient(s) R and/or the provider P have transitioned through the procedure.

In some embodiments, the therapeutic procedure may be any or a combination of counseling, assessment, diagnosis, therapy, and treatment undertaken by the provider on the recipient(s). The knowledge transfer procedure may be a transfer of any knowledge shared by the knowledge provider to the knowledge seeker.

In some embodiments, the processor 804 may gather the first and second set of brainwave activity signals either intermittently at defined intervals or may obtain the brainwave activity signals continuously before, during, and after the therapeutic/knowledge transfer procedure.

The state of mind of the provider P and/or the recipient(s) R may be any part or a combination of empathetic listening E mental state (also called beyond-mindfulness state or transcendental state), focused-attention F mental state, and an agitated A mental state. The efficacy of the therapeutic or knowledge transfer procedure may be higher when the recipient(s)′ R state of mind during and after the course of the procedure as indicated by the respective brainwave activity signals matches the brainwave activity signals of the provider P.

In some embodiments, the correlation of the first and second set of brainwave activity signals with the impact by the processor 804 enables identification of data patterns indicative of the states of mind through which the recipient(s) R and/or the provider P have transitioned through the procedure.

As used herein, a “processor” means one or more microprocessors, central processing units (CPUs), computing devices, microcontrollers, digital signal processors, or like devices or any combination thereof, regardless of their architecture. An apparatus that performs a process can include, e.g., a processor and those devices such as input devices and output devices that are appropriate to perform the process.

Processor(s) 804 can be (or include) any known processor, such as, but not limited to, an Intel® Itanium® or Itanium 2® processor(s), AMD® Opteron® or Athlon MP® processor(s), or Motorola® lines of processors, and the like. Communications port(s) 814 can be any of an RS-232 port for use with a modem-based dial-up connection, a 10/100 Ethernet port, a Gigabit port using copper or fiber, or a USB port, and the like. Communications port(s) 814 may be chosen depending on a network such as a Local Area Network (LAN), a Wide Area Network (WAN), a CDN, or any network to which the computer system 800 connects. The computer system 800 may be in communication with peripheral devices (e.g., display screen 816, input device(s) 818) via Input/Output (I/O) port 820. Some or all of the peripheral devices may be integrated into the computer system 800, and the input device(s) 818 may be integrated into the display screen 816 (e.g., in the case of a touch screen).

Main memory 806 can be Random Access Memory (RAM), or any other dynamic storage device(s) commonly known in the art. Read-only memory 808 can be any static storage device(s) such as Programmable Read-Only Memory (PROM) chips for storing static information such as instructions for the processor(s) 804. Mass storage 812 can be used to store information and instructions. For example, hard disks such as the Adaptec® family of Small Computer Serial Interface (SCSI) drives, an optical disc, an array of disks such as Redundant Array of Independent Disks (RAID), such as the Adaptec® family of RAID drives, or any other mass storage devices may be used.

Bus 802 communicatively couples processor(s) 804 with the other memory, storage, and communications blocks. Bus 802 can be a PCI/PCI-X, SCSI, a Universal Serial Bus (USB) based system bus (or other) depending on the storage devices used, and the like. Removable storage media 810 can be any kind of external hard-drives, floppy drives, IOMEGA® Zip Drives, Compact Disc-Read-Only Memory (CD-ROM), Compact Disc-Re-Writable (CD-RW), Digital Versatile Disk-Read Only Memory (DVD-ROM), etc.

Embodiments herein may be provided as one or more computer program products, which may include a machine-readable medium having stored thereon instructions, which may be used to program a computer (or other electronic devices) to perform a process. As used herein, the term “machine-readable medium” refers to any medium, a plurality of the same, or a combination of different media, which participate in providing data (e.g., instructions, data structures) which may be read by a computer, a processor, or a like device. Such a medium may take many forms, including but not limited to, non-volatile media, volatile media, and transmission media. Non-volatile media include, for example, optical or magnetic disks and other persistent memory. Volatile media include dynamic random access memory, which typically constitutes the main memory of the computer. Transmission media include coaxial cables, copper wire, and fiber optics, including the wires that comprise a system bus coupled to the processor. Transmission media may include or convey acoustic waves, light waves, and electromagnetic emissions, such as those generated during radio frequency (RF) and infrared (IR) data communications.

The machine-readable medium may include, but is not limited to, floppy diskettes, optical discs, CD-ROMs, magneto-optical disks, ROMs, RAMs, erasable programmable read-only memories (EPROMs), electrically erasable programmable read-only memories (EEPROMs), magnetic or optical cards, flash memory, or other types of media/machine-readable medium suitable for storing electronic instructions. Moreover, embodiments herein may also be downloaded as a computer program product, wherein the program may be transferred from a remote computer to a requesting computer by way of data signals embodied in a carrier wave or other propagation medium via a communication link (e.g., modem or network connection).

Various forms of computer-readable media may be involved in carrying data (e.g. sequences of instructions) to a processor. For example, data may be (i) delivered from RAM to a processor; (ii) carried over a wireless transmission medium; (iii) formatted and/or transmitted according to numerous formats, standards, or protocols; and/or (iv) encrypted in any of a variety of ways well known in the art.

As shown, main memory 806 is encoded with the application(s) 822 that support(s) the functionality as discussed herein (an application 822 may be an application that provides some or all of the functionality of one or more of the mechanisms described herein). Application(s) 822 (and/or other resources as described herein) can be embodied as software code such as data and/or logic instructions (e.g., code stored in the memory or on another computer-readable medium such as a disk) that supports processing functionality according to different embodiments described herein.

During operation of one embodiment, processor(s) 804 accesses main memory 810 via the use of bus 802 in order to launch, run, execute, interpret or otherwise perform the logic instructions of the application(s) 822. Execution of application(s) 822 produces processing functionality of the service(s) or mechanism(s) related to the application(s). In other words, the process(es) 824 represents one or more portions of the application(s) 822 performing within or upon the processor(s) 804 in the computer system 800.

It should be noted that in addition to the process(es) 824 that carries(carry) out operations as discussed herein, other embodiments herein include the application 822 itself (i.e., the un-executed or non-performing logic instructions and/or data). Application 822 may be stored on a computer-readable medium (e.g., a repository) such as a disk or in an optical medium. According to other embodiments, application 822 can also be stored in a memory type system such as in firmware, read-only memory (ROM), or, as in this example, as executable code within the main memory 810 (e.g., within Random Access Memory or RAM). For example, application 822 may also be stored in removable storage media 810, read-only memory 808, and/or mass storage device 812.

Those skilled in the art will understand that the computer system 800 can include other processes and/or software and hardware components, such as an operating system that controls allocation and use of hardware resources.

As discussed herein, embodiments of the present invention include various steps or operations. A variety of these steps may be performed by hardware components or may be embodied in machine-executable instructions, which may be used to cause a general-purpose or special-purpose processor programmed with the instructions to perform the operations. Alternatively, the steps may be performed by a combination of hardware, software, and/or firmware. The term “module” refers to a self-contained functional component, which can include hardware, software, firmware or any combination thereof.

One of ordinary skill in the art will readily appreciate and understand, upon reading this description, that embodiments of an apparatus may include a computer/computing device operable to perform some (but not necessarily all) of the described process.

Embodiments of a computer-readable medium storing a program or data structure include a computer-readable medium storing a program that, when executed, can cause a processor to perform some (but not necessarily all) of the described process.

Where a process is described herein, those of ordinary skill in the art will appreciate that the process may operate without any user intervention. In another embodiment, the process includes some human intervention (e.g., a step is performed by or with the assistance of a human).

As used herein, including in the claims, the phrase “one or more,” and includes the case of only one. Thus, e.g., the phrase “at least some ABCs” means “one or more ABCs”, and includes the case of only one ABC.

As used herein, including in the claims, a list may include only one item, and, unless otherwise stated, a list of multiple items need not be ordered in any particular manner. A list may include duplicate items. For example, as used herein, the phrase “a list of XYZs” may include one or more “XYZs”.

It should be appreciated that the words “first” and “second” in the description and claims are used to distinguish or identify, and not to show a serial or numerical limitation. Similarly, the use of letter or numerical labels (such as “(a)”, “(b)”, and the like) are used to help distinguish and/or identify, and not to show any serial or numerical limitation or ordering.

No ordering is implied by any of the labeled boxes in any of the flow diagrams unless specifically shown and stated. When disconnected boxes are shown in a diagram the activities associated with those boxes may be performed in any order, including fully or partially in parallel.

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and scope of the appended claims.

I claim:

1. A system comprising:
a processor operatively coupled with a memory, storing a set of instructions, which when processed by the processor, cause the processor to:
receive brainwave activity signals, before, during, and post a therapeutic/knowledge transfer procedure, from a first set of sensors configured in a primary headgear, a first set of brainwave activity signals associated with a provider P, said brainwave activity signals being indicative of state of mind of the provider P;
receive brainwave activity signals, before, during, and post the therapeutic/knowledge transfer procedure, from a second set of sensors configured in one or more secondary headgear(s), a second set of brainwave activity signals associated with one or more recipient(s) R, wherein, during the procedure, said provider performs a therapeutic/knowledge transfer procedure to evaluate the efficacy/impact on said recipient(s) R; and
correlate said first and second set of brainwave activity signals with said impact to determine efficacy of the therapeutic/knowledge transfer procedure performed by the provider P on the recipient(s) R.

2. The system as claimed in claim 1, wherein said therapeutic procedure is any or a combination of counseling, assessment, diagnosis, therapy, and treatment undertaken by the provider on the recipient(s); wherein said knowledge transfer procedure is a transfer of any knowledge shared by the knowledge provider to the knowledge seeker(s).

3. The system as claimed in claim 1, wherein said state of mind is any part or a combination of desirable mental states, including either empathetic listening/beyond-mindfulness state/transcendental state of mind E or a mindful/focused-attention F mental state, and/or an agitated A mental state.

4. The system as claimed in claim 1, wherein the efficacy of the therapeutic/knowledge transfer procedure is higher when the recipient(s)' R state of mind during and after the course of the procedure as indicated by the respective brainwave activity signals matches the empathic/transcendental E brainwave activity signals of the provider P.

5. The system as claimed in claim 1, wherein the step of correlating the first and second set of brainwave activity signals with said impact/efficacy enables identification of data patterns indicative of the states of mind through which the recipient(s) R have transitioned through the procedure provided by the provider P.

6. The system as claimed in claim 1, wherein the efficacy of the therapeutic/knowledge transfer procedure performed by the provider on the recipient(s) R can be objectified on any or a combination of a grade, range, scale, value, or a score.

7. The system as claimed in claim 1, wherein the headgear is based on an electroencephalogram (EEG) system and electromyography (EMG) system.

8. The system as claimed in claim 7, wherein the EEG system positions two or more scalp electrodes in a manner so as to gather brainwave measurements, said brainwaves being selected from Gamma brainwaves, Beta brainwaves, Alpha brainwaves, Theta brainwaves, and Delta brainwaves.

9. The system as claimed in claim 7, wherein the EMG system positions two or more electromyography (EMG) sensors so as to gather head muscle movement signals which may produce a false-positive brainwaves in the EEG sensors, which can be eliminated to produce true brain waves for correlation.

10. The system as claimed in claim 1, wherein the first and second set of brainwave activity signals are gathered either intermittently at defined intervals or are obtained continuously before, during, and after the therapeutic/knowledge transfer procedure.

11. The system as claimed in claim 1, wherein the procedure is started when the provider P is in an empathic listening E mental state which is desired state of a healer/provider P.

12. The system as claimed in claim 1, wherein during the procedure, the provider P attempts to bring the recipient(s) to a mindful/focused-attention F mental state and then to an empathic/transcendental E mental state which is desired state of healing.

13. The system as claimed in claim 1, wherein correlation between the first and second set of brainwave activity signals indicates emotional connection and/or synchronization of the recipient(s) R with the provider P.

14. The system as claimed in claim 1, wherein post the procedure, the system obtains information associated with a treatment/follow-up plan provided by the provider P to the recipient(s) R, and evaluates the said recipient(s) R at subsequent desired time intervals (follow-up procedures) to determine the impact/efficacy of the treatment/follow-up plan based on analysis of the brainwave activity signals associated with the said recipient(s) R at such respective time intervals.

15. A method comprising:
receiving brainwave activity signals, before, during, and post a therapeutic/knowledge transfer procedure, from a first set of sensors configured in a primary headgear, a first set of brainwave activity signals associated with a provider P, said brainwave activity signals being indicative of state of mind of the provider P;
receiving brainwave activity signals, before, during, and post the procedure, from a second set of sensors configured in one or more secondary headgear(s), a second set of brainwave activity signals associated with recipient(s) R, wherein, during the procedure, said provider P performs a therapeutic/knowledge transfer impact on said recipient(s) R; and
correlating said first and second set of brainwave activity signals with said impact to determine efficacy of the therapeutic/knowledge transfer procedure performed by the provider P on the recipient(s) R.

16. The method as claimed in claim 15, wherein said state of mind is any part or a combination of desirable, healing/therapeutic mental state (which may also be termed as an empathetic listening mental state or mindfulness and/or beyond mindfulness/transcendental state) E, and focused-attentive F mental state, or an undesirable or agitated A mental state.

17. The method as claimed in claim 15, wherein the efficacy of the therapeutic/knowledge transfer procedure is higher when the recipient(s)' R state of mind during and after the course of the procedure as indicated by the respective brainwave activity signals matches the brainwave activity signals of the provider P.

18. The method as claimed in claim 15, wherein the step of correlating the first and second set of brainwave activity signals with said impact enables identification of data patterns indicative of the states of mind through which the recipient(s) R have transitioned through the procedure provided by the provider P.

19. The method as claimed in claim 15, wherein the efficacy of the therapeutic/knowledge transfer procedure performed by the provider P on the recipient(s) R can be objectified on any or a combination of a grade, range, scale, value, or a score.

20. The method as claimed in claim 19, wherein the efficacy of the therapeutic/knowledge transfer procedure performed by the provider P on the recipient(s) R is determined based on one or more efficacy markers comprising any or a combination of:
an agitated A state of mind, wherein predominantly high beta waves along with very low gamma & alpha waves are measured;
a focused attention F state of mind, wherein predominantly high and sustained alpha waves are measured;
a mindful M state of mind, wherein predominantly high delta and theta waves are measured;
an empathic/transcendental E state of mind, wherein predominantly high and sustained gamma waves are measured; and
one or more categories based on a combination of any of Gamma brainwaves, Beta brainwaves, Alpha brainwaves, Theta brainwaves, and Delta brainwaves associated with the brain activity signals are measured.
